(19) United States
(12) Patent Application Publication
(10) Pub. No.: US 2023/0233376 A1
(43) Pub. Date: Jul. 27, 2023

(54) EYE DROP ILLUMINATION DEVICE AND METHOD

(71) Applicant: George L. Reynolds, Altona, IL (US)

(72) Inventor: George L. Reynolds, Altona, IL (US)

(21) Appl. No.: 18/149,936

(22) Filed: Jan. 4, 2023

Related U.S. Application Data
(60) Provisional application No. 63/266,987, filed on Jan. 21, 2022.

(51) Int. Cl. A61F 9/00

(52) U.S. Cl. CPC A61F 9/0026

(57) ABSTRACT

An eye drop dispenser includes a light absorbing element that emits light to facilitate properly aligning the eye drop dispenser with the eye while in the dark.

### CROSS-REFERENCE TO RELATED APPLICATION

The present application claims the benefit of the filing date of U.S. Provisional Application Ser. No. 63/266,987 filed on Jan. 21, 2022, which is incorporated herein by reference.

### BACKGROUND

Many people who suffer from dry eyes instill eye drops one or more times during the night. Since it is usually undesirable to turn on the lights, many people attempt (with the lights off and using trial and error in the dark) to instill eye drops anyway, but with the annoying consequence of getting eye drops on their faces, etc. before getting drops into their eyes.

### SUMMARY

The present invention provides a means for accurately instilling or administering eye drops while in the dark. The present invention provides an eye drop “aimer” whereby a light absorbing glow-in-the-dark ring, such as a visibly phosphorescing-ring, coaxially surrounds the dispensing end of a standard eye drop dispenser bottle (or tube or other dispenser) such that, even in the dark, the eye drop dispenser can be easily positioned directly above the center of the pupil of the eyeball before dispensing fluid from the eye drop dispenser bottle the eye drops.

Most over-the-counter and prescription eye drop dispenser bottles have standardized dispensing ends, as do most eye drop dispenser tubes and single-use dispensers, such that only a few different eye drop aimer configurations will accommodate most eye drop dispensers. The invention is adaptable to essentially any type of eye drop dispenser bottle, tube or other dispenser.

The light absorbing glow-in-the-dark element can be, for example, a luminescent or phosphorescent element made of any non-toxic, suitably clear and sufficiently elastic or non-elastic polymer, depending upon the application, that can be mixed with a suitable luminescent and/or phosphorescent material. However, embodiments contemplate any light emitting material capable of glowing in the dark.

A consideration when dispensing eye drops is that care needs to be exercised in order to avoid touching the eyeball with the fingers or any part of the eye drop dispenser. The invention eye drop aimer can be designed to warn the user that the eye drop dispenser is being held too closely to the eye, that is, when the phosphorescent or other light emitting ring cannot be seen when preparing to instill eye drops. Similarly if the ring-shaped light emitting element cannot be seen as a full circle this means that the dispenser is too close to the eye or is misaligned with the eye.

An additional benefit provided by the phosphorescence and/or luminescence of the eye drop aimer is that the eye drop dispenser can easily be seen in the dark so that the user does not have to grope in the dark for the eye drop dispenser.

### BRIEF DESCRIPTION OF THE DRAWINGS

FIG. 1 shows a sectional side view of an elastic luminescent and/or phosphorescent eye-drop-aimer poised above a typical commercial eye drop dispenser bottle prior to installation of the eye drop aimer onto the bottle.

FIG. 1A is a plan view of an elastic luminescent and/or phosphorescent eye drop aimer ring.

FIG. 2 is a partial sectional side view of an elastic eye drop aimer assembled onto an eye drop dispenser bottle.

FIG. 3 is a side view of an inverted eye drop dispenser with the cap removed, and with the dispenser being used to instill an eye drop.

FIG. 3A is a plan view of the approximate image as seen in the dark by the eye drop recipient as a result of what is shown in FIG. 3.

FIG. 4 shows an eye drop dispenser positioned too closely for safety relative to the eyeball.

FIG. 5 shows a misaligned eye drop dispenser.

FIG. 5A shows the image resulting from the misaligned dispenser of FIG. 5.

FIG. 6 shows an eye drop dispenser in which a luminescent and/or phosphorescent element has been cast or molded onto an eye drop dispenser.

FIG. 7 shows an eye drop dispenser in which luminescent and/or phosphorescent paint or heat-shrinkable tubing has been applied to the dispenser body.

FIG. 8 illustrates an eye drop dispenser with an alternative cap design.

FIG. 9 illustrates the alternative cap design with the cap removed.

FIG. 10 illustrates the nozzle portion of a typical eye drop dispenser.

### DETAILED DESCRIPTION OF THE ILLUSTRATED EMBODIMENTS

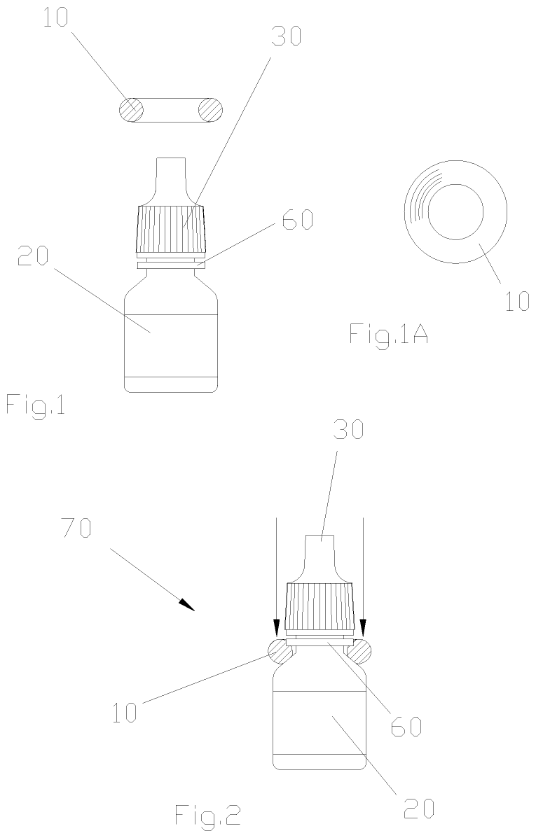

FIG. 1 illustrates light absorbing glow-in-the-dark emitting eye drop aimer ring 10 (hereinafter referred to as “eye drop aimer ring”) poised above a typical eye drop dispenser bottle 20. Eye drop aimer ring 10 can be elastic or non-elastic according to the application. The inside diameter of eye drop aimer ring 10 is smaller than an outer diameter of rim 60 so that eye drop aimer ring 10 will be retained on eye drop dispenser bottle 20 when aimer ring 10 is assembled onto dispenser bottle 20. Eye drop aimer ring 10 is composed of light absorbing material that is capable of glowing in the dark.

In an embodiment, eye drop aimer ring 10 is composed of any appropriate material mixed with a suitable phosphorescent dye such that in the absence of ambient light eye drop aimer ring 10 will glow sufficiently so that the eye drop recipient will be able to clearly see the eye drop aimer ring 10 in the dark. Other embodiments contemplate eye drop aimer ring including any luminescent material.

FIG. 1A is a plan view of eye drop aimer ring 10 showing the circular nature of eye drop aimer ring 10. However it will be obvious that the eye drop aimer ring 10 can be of any geometric shape that will permit securing the eye drop aimer ring 10 to this type or other dispenser types, and that such eye drop aimer rings 10 will facilitate aligning the dispenser for accurately instilling or administering eye drops.

FIG. 2 is a partial sectional side view showing the eye drop bottle assembly 70 after eye drop aimer ring 10 has been urged downwardly, first elastically expanding over, and then contracting under, rim 60 of typical eye drop dispensing bottle 20, securing eye drop aimer ring 10 under rim 60 of eye drop dispenser bottle 20. Cap 30 is kept in place while installing light emitting eye drop aimer ring 10, thus protecting the dispenser nozzle from contamination. If, as in this case, eye drop aimer ring 10 is elastic, it can be removed and be reused indefinitely.

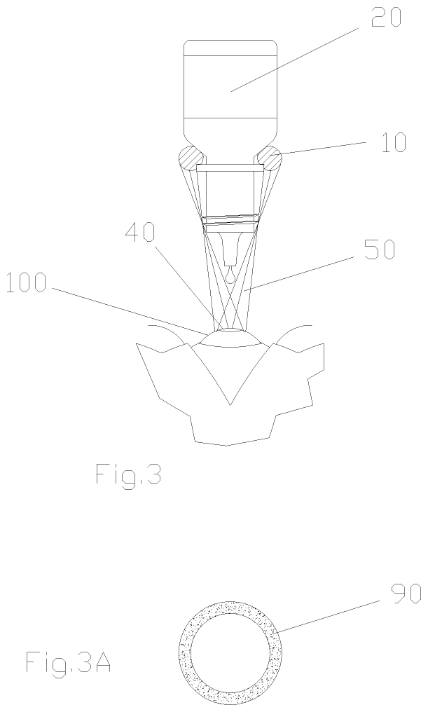

FIG. 3 shows eye drop dispenser 20 in which cap 30 has been removed, and eye drop dispenser bottle 20 has been inverted so that the glowing or phosphorescence in eye drop aimer ring 10 is clearly visible in the dark to the user, thereby aiding positioning of eye drop dispenser bottle 20 over the center of the eye 100 of the eye drop recipient for instilling eye drops.

With eye drop dispenser bottle 20 positioned as shown it can be seen that light paths 50 from light emitting eye drop aimer ring 10 impinge on eyeball pupil 40. Eye drop aimer ring is visible as a complete circle around eye drop dispenser bottle 20 so that the eye drop recipient can see that eye drop dispenser 20 is centered over the recipient's eye, and that the eye drop dispenser 20 is at a safe distance from the eye 100.

FIG. 3A illustrates the image 90 of aimer ring 10 formed in the vision of the eye drop recipient when eye drop bottle 20 of FIG. 3 is properly/safely positioned for instilling eye drops.

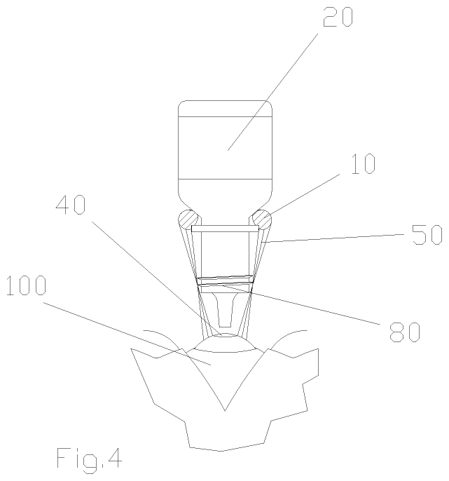

FIG. 4 shows the dispenser 20 unsafely close to the eye 100. With the dispenser in this proximity it can be seen that the light paths 50 do not enter the pupil 40 of the eye 100. With eye drop dispenser 20 positioned as in FIG. 4, then eye drop aimer ring 10 will not be visible or will only be partially visible, alerting the recipient to move the dispenser farther away from his/her eyeball.

FIG. 5 shows eye drop dispenser 20 slightly laterally displaced relative to the center of the eye 100, thus misaligning the dispenser. In this case threads 80 of bottle 20 block part of the light path 50 to the eye so the image formed in the eye will appear eccentric as in FIG. 5A.

FIG. 5A illustrates the approximate image 90 formed in the vision of the eye drop recipient when the dispenser 20 is displaced as shown in FIG. 5, thus alerting the recipient to properly align the dispenser.

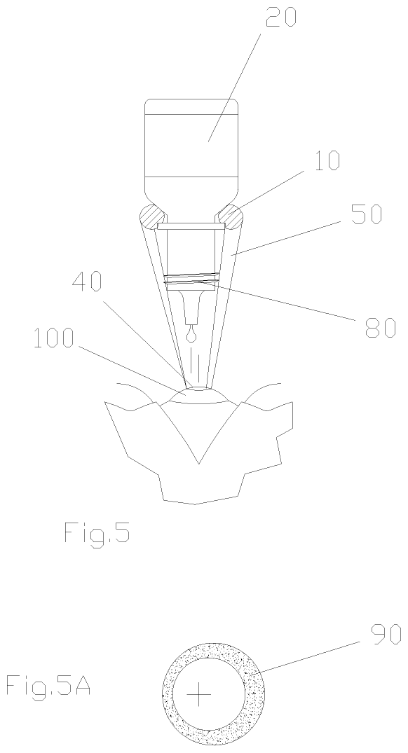

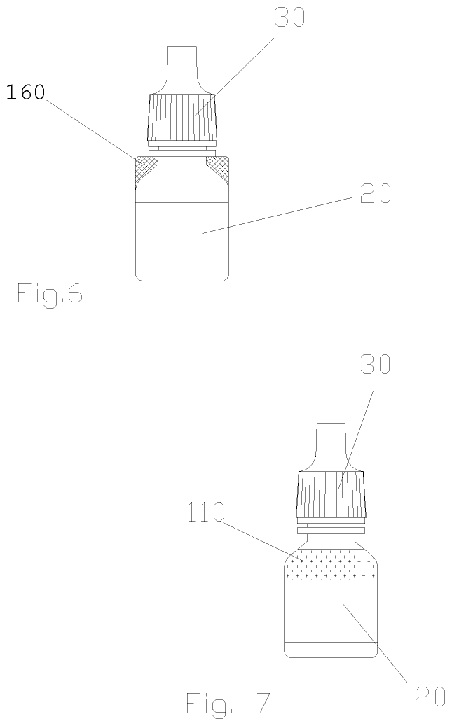

FIG. 6 shows an eye drop dispenser 20 in which a luminescent and/or phosphorescent cast or molded aimer ring 160 has been incorporated.

The entire dispenser bottle 20 can also be molded of a luminescent, phosphorescent and/or florescent plastic, provided that such plastic is non-toxic. However this would eliminate the safety feature of alerting the user if positioning the eye drop dispenser too closely to the eye as the entire bottle would glow, thereby eliminating the eccentric appearance of a misaligned dispenser bottle.

FIG. 7 shows an eye drop dispenser bottle 20 in which luminescent and/or phosphorescent paint 110 has been applied to the dispenser bottle body 20. Heat-shrinkable luminescent, and/or phosphorescent tubing can also be applied instead of, or in combination with luminescent, and/or phosphorescent paint.

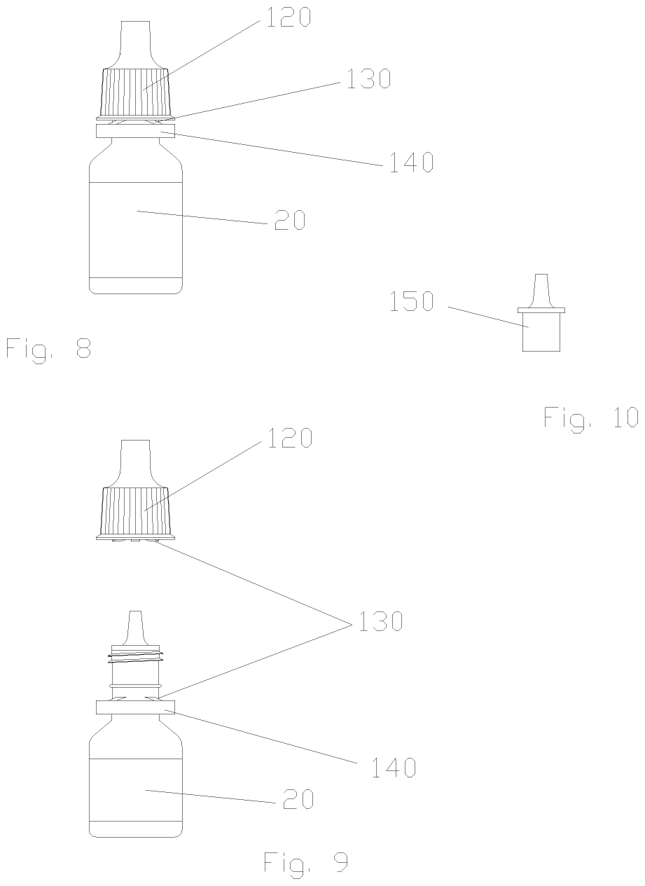

FIG. 8 illustrates an alternative eye drop dispenser style in which the unopened dispenser is provided with cap 120 secured to cap base 140 by integral, frangible bridges 130. Integral cap 120 with bridges 130 and cap base 140 can be molded of luminescent and/or phosphorescent material.

FIG. 9 illustrates the eye drop dispenser of FIG. 8, but integral cap 120 has been unscrewed, thereby breaking bridges 130, leaving luminescent and/or phosphorescent cap base 140 on eye drop dispenser bottle 20 with light emitting cap base 140 serving as the eye drop aiming ring. In this scenario cap 120 would also remain clearly visible in the dark if laid aside during instilling the eye drops.

FIG. 10 shows a typical eye drop dispensing nozzle 150 which can also be made of luminescent and/or phosphorescent material.

### LIST OF ELEMENTS

10 eye drop aimer ring
20 eye drop dispenser bottle
30 cap
40 eyeball pupil
50 light path
60 rim
70 eye drop bottle assembly
80 threads
90 image
100 eye
110 paint
120 integral cap
130 bridges
140 cap base
150 nozzle
160 cast or molded aimer ring

What is claimed is:

1. An eye drop dispenser for dispensing a fluid to an eye, the eye drop dispenser comprising:
an eye drop dispenser bottle for containing the fluid, the eye drop dispenser bottled configured to dispense the fluid as eye drops when inverted; and
an eye drop aimer ring extending around the eye drop dispenser bottle, wherein the eye drop aimer ring is comprised of light absorbing material that emits light in the dark that is visible by the eye to align the inverted eye drop dispenser bottle with the eye.

2. The eye drop dispenser of claim 1, wherein the eye drop aimer ring is circular.

3. The eye drop dispenser of claim 1, wherein the eye drop aimer ring is removable from the eye drop dispenser bottle.

4. The eye drop dispenser of claim 3, wherein the eye drop aimer ring is elastically deformable to expand for positioning the eye drop aimer ring over the eye drop dispenser bottle and to thereafter contract to secure the eye drop aimer ring to the eye drop dispenser bottle.

5. The eye drop dispenser of claim 4, wherein the eye drop dispenser bottle includes a rim and the eye drop aimer ring expands to pass over the rim and contracts under the rim to secure the eye drop aimer ring to the eye drop dispenser bottle.

6. The eye drop dispenser of claim 5, wherein the eye drop aimer ring includes an inner diameter, the rim includes an outer diameter, and the inner diameter of the eye drop aimer ring is less than the outer diameter of the rim.

7. The eye drop dispenser of claim 1, wherein:
the eye drop dispenser bottle includes a base and a cap secured to the base; and
the base forms the eye drop aimer ring.

8. The eye drop dispenser of claim 7, wherein the cap is secured to the base with integral, frangible bridges.

9. The eye drop dispenser of claim 8, wherein the cap, base, and frangible bridges are each comprised of light absorbing material that emits light in the dark.

10. The eye drop dispenser of claim 1, wherein the eye drop aimer ring is formed by a light absorbing paint applied around the eye drop dispenser bottle.

11. The eye drop dispenser of claim 1, wherein the eye drop aimer ring is formed by a light absorbing tape applied around the eye drop dispenser bottle.

12. The eye drop dispenser of claim 1, wherein the eye drop aimer ring is cast or molded with the eye drop dispenser bottle.

13. The eye drop dispenser of claim 1, wherein the eye drop dispenser bottle includes a nozzle, and the nozzle is comprised of light absorbing material that emits light in the dark.

14. The eye drop dispenser of claim 1, wherein the eye drop dispenser bottle is aligned with the eye when the eye drop aimer ring is visible as a complete circle around the eye drop dispenser bottle.

15. The eye drop dispenser of claim 1, wherein the eye drop aimer ring is comprised of a phosphorescent dye mixed with an elastically deformable material.

16. A method for dispensing eye drops from an eye drop dispenser into an eye in the dark, the method comprising:
inverting the eye drop dispenser over the eye while in the dark; and
aligning the eye drop dispenser with the eye, wherein aligning the eye drop dispenser includes manipulating the eye drop dispenser until light emitted from a light absorbing element on the inverted eye drop dispenser impinges on the eye.

17. The method of claim 16, wherein the inverted eye drop dispenser is aligned with the eye when the light absorbing element is visible by the eye as a complete circle around the eye drop dispenser.

18. The method of claim 17, wherein the light absorbing element being visible as the complete circle further indicates the eye drop dispenser is sufficiently spaced away from the eye.

19. The method of claim 17, wherein the inverted eye drop dispenser is misaligned with the eye when the light absorbing element is visible as an eccentric image by the eye.

20. The method of claim 16, wherein the light absorbing element is a luminescent ring extending around the eye drop dispenser.
